(19) United States
(12) Patent Application Publication
(10) Pub. No.: US 2024/0361267 A1
(43) Pub. Date: Oct. 31, 2024

(54) HYDROGEN DETECTION METHOD, DRIVE CIRCUIT, AND HYDROGEN DETECTION DEVICE

(71) Applicant: Nuvoton Technology Corporation Japan, Kyoto (JP)

(72) Inventors: Koji KATAYAMA, Nara (JP); Kazunari HOMMA, Gifu (JP); Ken KAWAI, Osaka (JP)

(21) Appl. No.: 18/766,297

(22) Filed: Jul. 8, 2024

Related U.S. Application Data
(63) Continuation of application No. PCT/JP2022/047403, filed on Dec. 22, 2022.

(30) Foreign Application Priority Data
Jan. 17, 2022 (JP) 2022-005362

(51) Int. Cl. G01N 27/04; G01N 33/00

(52) U.S. Cl. CPC G01N 27/045; G01N 33/005

(57) ABSTRACT

A hydrogen detection method is a method performed using a hydrogen sensor that includes: a metal oxide layer; a second electrode that is in surface contact with the metal oxide layer; a first terminal connected to the second electrode; and a second terminal connected to the second electrode. The hydrogen detection method includes: applying a first voltage pulse between the first terminal and the second terminal to cause a chemical reaction of the metal oxide layer to hydrogen; and applying a second voltage pulse between the first terminal and the second terminal after the applying of the first voltage pulse, to detect a change in resistance between the first terminal and the second terminal. The amplitude of the second voltage pulse is smaller than the amplitude of the first voltage pulse.

### CROSS REFERENCE TO RELATED APPLICATIONS

This is a continuation application of PCT International Application No. PCT/JP2022/047403 filed on Dec. 22, 2022, designating the United States of America, which is based on and claims priority of Japanese Patent Application No. 2022-005362 filed on Jan. 17, 2022. The entire disclosures of the above-identified applications, including the specifications, drawings and claims are incorporated herein by reference in their entirety.

### FIELD

The present disclosure relates to: a hydrogen detection method that is performed using a hydrogen sensor; a drive circuit that drives the hydrogen sensor; and a hydrogen detection device including the hydrogen sensor and the drive circuit.

### BACKGROUND

Patent Literature (PTL) 1 and 2 disclose conventional gas sensors that detect gas molecules containing hydrogen atoms. According to PTL 1 and 2, however, the performance in detection of low-concentrated hydrogen is low in particular. In view of this, PTL 3 discloses an element structure of a hydrogen sensor or a hydrogen detection method for improved performance in detection of low-concentrated hydrogen.

### CITATION LIST

### Patent Literature

PTL 1: WO2017/037984

PTL 2: Japanese Unexamined Patent Application Publication No. 2017-173307

PTL 3: WO2021/210453

### SUMMARY

### Technical Problem

PTL 3 discloses a method of passing current and, at the same time, detecting a decrease in electric resistance (hereinafter also simply referred to as “resistance”). With the hydrogen sensor according to PTL 3, however, passage of large current for enhancing the performance in detection of low-concentrated hydrogen in particular causes a change in the state of the element sensor and also a change in the current value even in the state of no hydrogen (base state), thus not allowing accurate detection of hydrogen concentration.

In view of the above, the present disclosure aims to provide a hydrogen detection method, a drive circuit, and a hydrogen detection device for stabilizing the base state and detecting hydrogen concentration more accurately than conventional techniques.

In addition, the present disclosure also aims to provide a hydrogen detection method, a drive circuit, and a hydrogen detection device for stabilizing the base state and detecting a wide range of hydrogen concentration.

### Solution to Problem

A hydrogen detection method according to an aspect of the present disclosure is a hydrogen detection method that is performed using a hydrogen sensor, the hydrogen sensor including: a metal oxide layer; an electrode that is in surface contact with the metal oxide layer; a first terminal connected to the electrode; and a second terminal connected to the electrode, the hydrogen detection method including: applying a first voltage pulse between the first terminal and the second terminal to cause a chemical reaction of the metal oxide layer to hydrogen; and applying a second voltage pulse between the first terminal and the second terminal after the applying of the first voltage pulse, to detect a change in resistance between the first terminal and the second terminal, wherein an amplitude of the second voltage pulse is smaller than an amplitude of the first voltage pulse.

A drive circuit according to an aspect of the present disclosure is a drive circuit that drives a hydrogen sensor, the hydrogen sensor including: a metal oxide layer; an electrode that is in surface contact with the metal oxide layer; a first terminal connected to the electrode; and a second terminal connected to the electrode, the drive circuit including: an applier that applies a first voltage pulse between the first terminal and the second terminal to cause a chemical reaction of the metal oxide layer to hydrogen, and then applies a second voltage pulse between the first terminal and the second terminal; and a detector that detects resistance between the first terminal and the second terminal while the second voltage pulse is applied between the first terminal and the second terminal, wherein an amplitude of the second voltage pulse is smaller than an amplitude of the first voltage pulse.

A hydrogen detection device according to an aspect of the present disclosure includes: a hydrogen sensor that includes a metal oxide layer, an electrode that is in surface contact with the metal oxide layer, a first terminal connected to the electrode, and a second terminal connected to the electrode; and the above-described drive circuit that drives the hydrogen sensor.

### Advantageous Effects

The hydrogen detection method, the drive circuit, and the hydrogen detection device according to the present disclosure can stabilize the base state and more accurately detect hydrogen concentration than conventional techniques.

In addition, the hydrogen detection method, the drive circuit, and the hydrogen detection device according to the present disclosure can stabilize the base state and detect a wide range of hydrogen concentration.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1A is a cross-sectional view illustrating a configuration example of a hydrogen sensor according to Embodiments 1 and 2.

FIG. 1B is a top view illustrating the configuration example of the hydrogen sensor according to Embodiments 1 and 2.

FIG. 2 is a block diagram illustrating a configuration example of a hydrogen detection device that includes a drive circuit and a hydrogen sensor and performs a hydrogen detection method according to Embodiments 1 and 2.

FIG. 3 is a block diagram illustrating a configuration example of a hydrogen detection device that includes a drive circuit and a hydrogen sensor and performs a hydrogen detection method using a bridge circuit according to Embodiments 1 and 2.

FIG. 4 is a block diagram illustrating a configuration example of a hydrogen detection device that includes a drive circuit and a hydrogen sensor and performs a hydrogen detection method using a bridge circuit according to another form of Embodiments 1 and 2.

FIG. 5A is a flowchart illustrating a hydrogen detection method according to a comparative example of Embodiment 1.

FIG. 5B is a diagram illustrating a voltage application pattern of the hydrogen detection method according to the comparative example illustrated in FIG. 5A when time is put on the horizontal axis.

FIG. 6 is a diagram illustrating experimental results obtained by performing the hydrogen detection method according to the comparative example illustrated in FIG. 5A and FIG. 5B.

FIG. 7A is a flowchart illustrating the hydrogen detection method according to Embodiment 1.

FIG. 7B is a diagram illustrating a voltage application pattern of the hydrogen detection method illustrated in FIG. 7A when time is put on the horizontal axis.

FIG. 8 is a diagram illustrating experimental results on dependence on the amplitude of a first voltage pulse in the hydrogen detection method according to Embodiment 1.

FIG. 9 is a diagram illustrating experimental results on dependence on the pulse width of the first voltage pulse in the hydrogen detection method according to Embodiment 1.

FIG. 10A is a timing diagram for describing time tint between the end of application of the first voltage pulse and the start of application of a second voltage pulse in the hydrogen detection method according to Embodiment 1.

FIG. 10B is a diagram illustrating an experimental result (an amount of change in differential voltage dV) obtained when time tint in FIG. 10A is changed.

FIG. 11 is a diagram for describing experiments related to the hydrogen detection method according to Embodiment 2.

FIG. 12 is a diagram illustrating relationships between a range of hydrogen concentration and an amount of change in differential voltage in the hydrogen detection method according to Embodiment 2.

FIG. 13 is a flowchart illustrating the hydrogen detection method according to Embodiment 2.

### DESCRIPTION OF EMBODIMENTS

Hereinafter, exemplary embodiments will be specifically described with reference to the drawings.

Note that the embodiments described below each show a general or specific example.

The numerical values, shapes, materials, constituent elements, the arrangement and connection of the constituent elements, steps, the processing order of the steps etc. illustrated in the embodiments described below are mere examples, and are not intended to limit the present disclosure. Also, the expression “detect resistance or a change in resistance” or the like includes not only direct detection of resistance or a change in resistance but also indirect detection of resistance or a change in resistance through detection of physical quantities other than resistance, such as voltage or current.

### Embodiment 1

First, a hydrogen sensor, a hydrogen detection method, a drive circuit, and a hydrogen detection device according to Embodiment 1 will be described.

### [1.1 Configuration of Hydrogen Sensor 1]

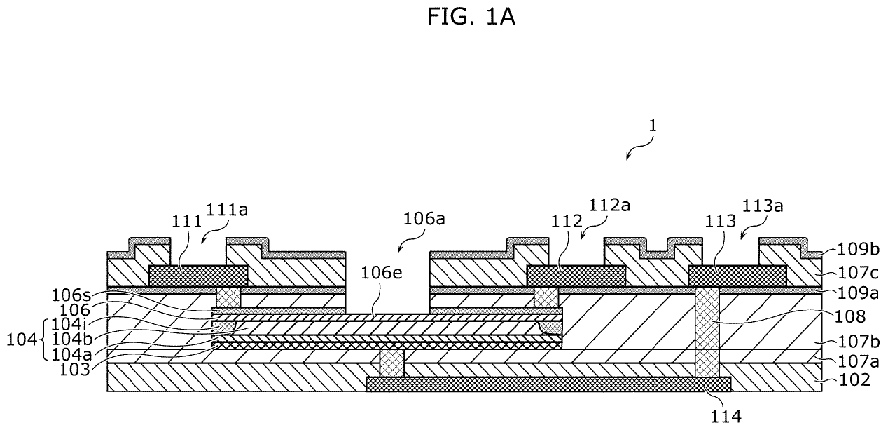

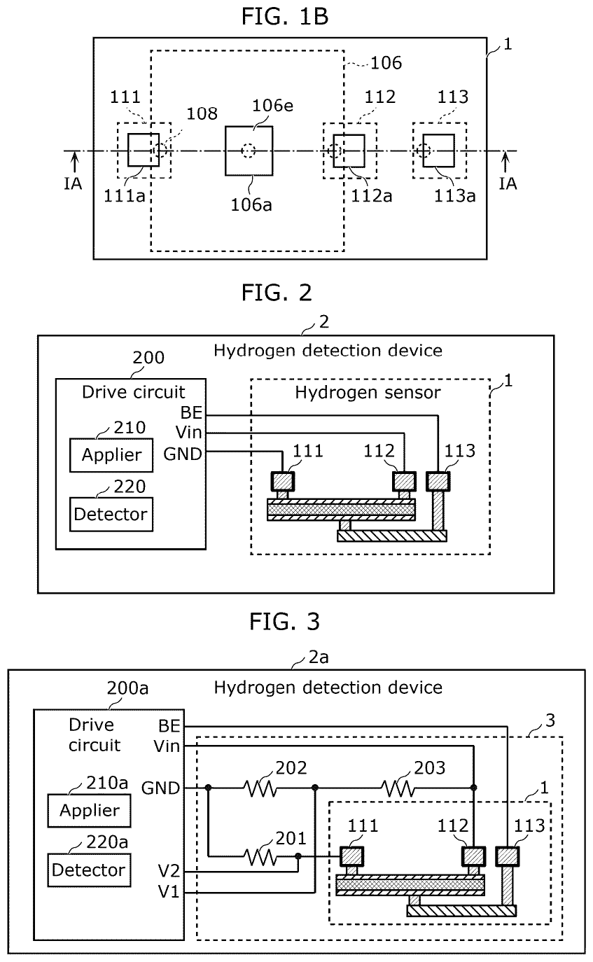

FIG. 1A is a cross-sectional view illustrating a configuration example of hydrogen sensor 1 according to Embodiment 1. FIG. 1B is a top view illustrating the configuration example of hydrogen sensor 1 according to Embodiment 1. Note that FIG. 1A illustrates a schematic cross section along line IA-IA of FIG. 1B, viewed in the arrow direction.

As illustrated in FIG. 1A and FIG. 1B, key components of hydrogen sensor 1 include first electrode 103, metal oxide layer 104, second electrode 106, first terminal 111, second terminal 112, and third terminal 113. The key components of hydrogen sensor 1 are covered by insulating film 102, insulating films 107a through 107c, and insulating films 109a and 109b. These insulating films, however, have openings 106a, 111a, 112a, and 113a.

First electrode 103 is a planar electrode and has two surfaces. Of the two surfaces of first electrode 103, one surface (i.e., the upper surface of first electrode 103 in FIG. 1A) is in contact with metal oxide layer 104, and the other surface (i.e., the lower surface of first electrode 103 in FIG. 1A) is in contact with insulating film 107b and via 108. In FIG. 1B, first electrode 103 is in a rectangular shape of the same size as that of second electrode 106. First electrode 103 may include, for example, a material having a standard electrode potential lower than that of metals forming metal oxides, such as tungsten, nickel, tantalum, titanium, aluminum, tantalum nitride, or titanium nitride. The higher the value of the standard electrode potential is, the more resistant to oxidation the material is. First electrode 103 in FIG. 1A is formed with, for example, tantalum nitride (TaN) or titanium nitride (TiN), or laminations thereof.

Metal oxide layer 104 is sandwiched between a surface of first electrode 103 and a surface of second electrode 106 facing each other, is formed with a metal oxide serving as a gas-sensitive resistance film, and has resistance that reversibly changes according to the presence and absence of a hydrogen-containing gas in a gas in contact with second electrode 106. It suffices so long as metal oxide layer 104 has a property that its resistance is changed by hydrogen. Metal oxide layer 104 is formed with an oxygen-deficient metal oxide (i.e., a metal oxide having a degree of oxygen deficiency). As the base metal of metal oxide layer 104, at least one of the following may be selected: aluminum (Al) or a transition metal such as tantalum (Ta), hafnium (Hf), titanium (Ti), zirconium (Zr), niobium (Nb), tungsten (W), nickel (Ni), or iron (Fe). Since transition metals can take on plural oxidation states, different resistance states can be realized through redox reactions. Here, the “degree of oxygen deficiency” of a metal oxide is the ratio of deficiency of oxygen in the metal oxide to the amount of oxygen in an oxide having a stoichiometric composition composed of the same elements as those of the metal oxide. Here, the oxygen deficiency is a value obtained by subtracting the amount of oxygen in the metal oxide from the amount of oxygen in the metal oxide having a stoichiometric composition. If there can be two or more metal oxides having stoichiometric compositions composed of the same elements as those of the metal oxide, the degree of oxygen deficiency of the metal oxide is defined based on one of the two or more metal oxides having stoichiometric compositions that has the highest resistance. Metal oxides having stoichiometric compositions are more stable and higher in resistance than metal oxides having other compositions. For example, when the base metal of metal oxide layer 104 is tantalum (Ta), the oxide having a stoichiometric composition as defined above is Ta₂O₅, so metal oxide layer 104 can be expressed as TaO_{2.5}. The degree of oxygen deficiency of TaO_{2.5} is 0% and the degree of oxygen deficiency of TaO_{1.5} is (2.5−1.5)/2.5=40%. The degree of oxygen deficiency of a metal oxide with excess oxygen is a negative value. Note that in the present disclosure, the degree of oxygen deficiency can take a positive value, 0, or a negative value unless otherwise noted. An oxide with a low degree of oxygen deficiency has high resistance because it is closer to an oxide having a stoichiometric composition, whereas an oxide with a high degree of oxygen deficiency has low resistance because it is closer to the metal included in the oxide.

Metal oxide layer 104 illustrated in FIG. 1A includes: first layer 104a in contact with first electrode 103; second layer 104b in contact with first layer 104a and second electrode 106; and isolation layer 104i. The degree of oxygen deficiency of second layer 104b is lower than that of first layer 104a. For example, first layer 104a is TaOₓ. Second layer 104b is Ta₂O₅ whose degree of oxygen deficiency is lower than that of first layer 104a. Metal oxide layer 104 includes isolation layer 104i at the perimeter in plan view of first electrode 103.

Here, plan view means viewing hydrogen sensor 1 according to the present disclosure from a viewpoint in the layer-stacking direction in FIG. 1A. In other words, plan view means viewing from a viewpoint in the direction normal to any of the surfaces of, for example, first electrode 103 and second electrode 106 that are planar. For example, plan view refers to viewing the top surface of hydrogen sensor 1 illustrated in FIG. 1B.

The resistance of such metal oxide layer 104 decreases according to a hydrogen-containing gas that comes into contact with second electrode 106. In detail, when a hydrogen-containing gas is present in a detection-target gas, hydrogen atoms are dissociated from the hydrogen-containing gas in second electrode 106. The dissociated hydrogen atoms enter metal oxide layer 104 and form impurity levels. In particular, the dissociated hydrogen atoms concentrate in the vicinity of the interface with second electrode 106, making the apparent thickness of second layer 104b smaller. As a result, the resistance of metal oxide layer 104 decreases.

Second electrode 106 is a planar electrode with hydrogen dissociability and has two surfaces. Of the two surfaces of second electrode 106, one surface (i.e., the lower surface of second electrode 106 in FIG. 1A) is in contact with metal oxide layer 104, and the other surface (i.e., the upper surface of second electrode 106 in FIG. 1A) is in contact with metal layer 106s and the outside air. Second electrode 106 has, in aperture 106a, exposed portion 106e that is exposed to the outside air. Second electrode 106 is formed with, for example, a material that catalyzes dissociation of hydrogen atoms from gas molecules having hydrogen atoms, such as platinum (Pt), iridium (Ir), palladium (Pd), or nickel (Ni), or an alloy containing at least one of these. It is assumed that second electrode 106 in FIG. 1A is platinum (Pt). Two terminals, namely first terminal 111 and second terminal 112, are connected to second electrode 106.

First terminal 111 is connected to second electrode 106 through via 108.

Second terminal 112 is connected to second electrode 106 through via 108. First terminal 111 and second terminal 112 are connected, via openings 111a and 112a, respectively, to an external drive circuit that drives hydrogen sensor 1.

As illustrated in FIG. 1B, first terminal 111 and second terminal 112 are disposed with exposed portion 106e being interposed therebetween in plan view of second electrode 106.

With first terminal 111 and second terminal 112 disposed in this manner, application of a predetermined voltage pulse between first terminal 111 and second terminal 112 causes passage of current through exposed portion 106e of second electrode 106, that is, causes current to flow through exposed portion 106e. The passage of current through exposed portion 106e of second electrode 106 is considered to activate the hydrogen dissociation by exposed portion 106e. Note that the predetermined voltage may be voltages that are opposite to each other in polarity.

In hydrogen sensor 1, the resistance between first terminal 111 and second terminal 112 changes when gas molecules containing hydrogen atoms come into contact with exposed portion 106e during the passage of current through exposed portion 106e. By the above-described drive circuit detecting this change in resistance, gas molecules containing hydrogen atoms are detected.

Third terminal 113 is connected to first electrode 103 via opening 113a, via 108, wiring 114, and via 108. Third terminal 113 is connected, via opening 113a, to the external drive circuit that drives hydrogen sensor 1.

In hydrogen sensor 1, the resistance between first electrode 103 and second electrode 106 changes when gas molecules containing hydrogen atoms come into contact with exposed portion 106e during the passage of current through exposed portion 106e. In other words, in hydrogen sensor 1, the resistance state between first terminal 111 or second terminal 112 and third terminal 113 changes when gas molecules containing hydrogen atoms come into contact with exposed portion 106e during the passage of current through exposed portion 106e. Gas molecules containing hydrogen atoms are detected also through detection, by the above-described drive circuit, of the change in the resistance state.

Note that insulating film 102, insulating films 107a through 107c, and insulating films 109a and 109b that cover the key components of hydrogen sensor 1 are formed with a silicon oxide film or a silicon nitride film, for example.

Metal layer 106s is formed on the upper surface of second electrode 106 except for opening 106a. Metal layer 106s includes, for example, TiAlN as the material and is formed as an etching stopper for forming vias 108, but is not essential.

The laminate of first electrode 103, metal oxide layer 104, and second electrode 106 is a structure that can be used as a storage element of resistive random access memory (ReRAM). The storage element of the resistive random access memory is a digital storage element which uses two of possible states that metal oxide layer 104 can take, i.e., a high-resistance state and a low-resistance state. Hydrogen sensor 1 according to the present disclosure uses the high-resistance state among the possible states of metal oxide layer 104.

FIG. 1A illustrates an example of metal oxide layer 104 having a two-layer configuration with first layer 104a that includes TaOₓ as the material and second layer 104b that includes, as the material, Ta₂O₅ whose degree of oxygen deficiency is low. However, metal oxide layer 104 may have a one-layer configuration having, as the material, TaOₓ or Ta₂O₅ whose degree of oxygen deficiency is low.

### [1.2 Hydrogen Detection Device]

Next, a hydrogen detection device including hydrogen sensor 1 will be described.

FIG. 2 is a block diagram illustrating a configuration example of hydrogen detection device 2 that includes drive circuit 200 and hydrogen sensor 1 and performs a hydrogen detection method according to Embodiment 1. In FIG. 2, hydrogen detection device 2 includes drive circuit 200 and hydrogen sensor 1. Drive circuit 200 is connected to hydrogen sensor 1 via at least three wires connected to first terminal 111, second terminal 112, and third terminal 113 of hydrogen sensor 1. Drive circuit 200 functionally includes: applier 210 that applies a first voltage pulse between first terminal 111 and second terminal 112 to cause a chemical reaction of metal oxide layer 104 to hydrogen and then applies a second voltage pulse between first terminal 111 and second terminal 112; and detector 220 that detects resistance between first terminal 111 and second terminal 112 while the second voltage pulse is applied between first terminal 111 and second terminal 112. As the hardware configuration, drive circuit 200 includes: a microcontroller including a central processing unit (CPU), read-only memory (ROM), random-access memory (RAM), and an analog-digital (AD) converter; a pulse generation circuit; a current measurement circuit; and a control circuit, for example.

Applier 210 of drive circuit 200 in hydrogen detection device 2 applies a predetermined voltage between first terminal 111 and second terminal 112. For example, first terminal 111 is set to GND (0 V) and voltage Vin is applied to second terminal 112. This allows a current of, for example, several milliamperes to several tens of milliamperes to flow through exposed portion 106e of second electrode 106. Detector 220 of drive circuit 200 measures the value of current flowing between first terminal 111 and second terminal 112 or an amount of change in the value of current flowing between first terminal 111 and second terminal 112 and calculates the hydrogen concentration using a predetermined conversion equation between the amount of change and hydrogen concentration.

The amount of change in current value caused by hydrogen is minute on the order of microamperes at a current of several milliamperes to several tens of milliamperes, and hydrogen detection device 2a as illustrated in the block diagram in FIG. 3 may be used. FIG. 3 is a block diagram illustrating a configuration example of a hydrogen detection device that includes a drive circuit and a hydrogen sensor and performs a hydrogen detection method using a bridge circuit according to Embodiment 1.

In FIG. 3, hydrogen detection device 2a includes bridge circuit 3 in which series-connected hydrogen sensor 1 and resistor 201 and series-connected resistor 202 and resistor 203 are connected in parallel. In general, the bridge circuit is suitable for detecting minute changes in resistance. Resistor 201 and resistor 202 may have the same resistance and hydrogen sensor 1 and resistor 203 may have the same resistance.

Applier 210a of drive circuit 200a sets the terminal of resistor 201 of bridge circuit 3 not connected to hydrogen sensor 1 to GND (0V) and applies voltage Vin to second terminal 112 of hydrogen sensor 1.

Accordingly, it is possible to pass a current of, for example, several milliamperes to several tens of milliamperes through exposed portion 106e of second electrode 106. Detector 220a of drive circuit 200 measures differential voltage dV (=V2−V1) between voltage V2 at the connection terminal of hydrogen sensor 1 and resistor 201 and voltage V1 at the connection terminal of resistor 202 and resistor 203 (hereinafter “differential voltage dV” is also simply referred to as a “differential voltage”) or an amount of change in the differential voltage, and calculates the hydrogen concentration using a predetermined conversion equation between the amount of change and hydrogen concentration.

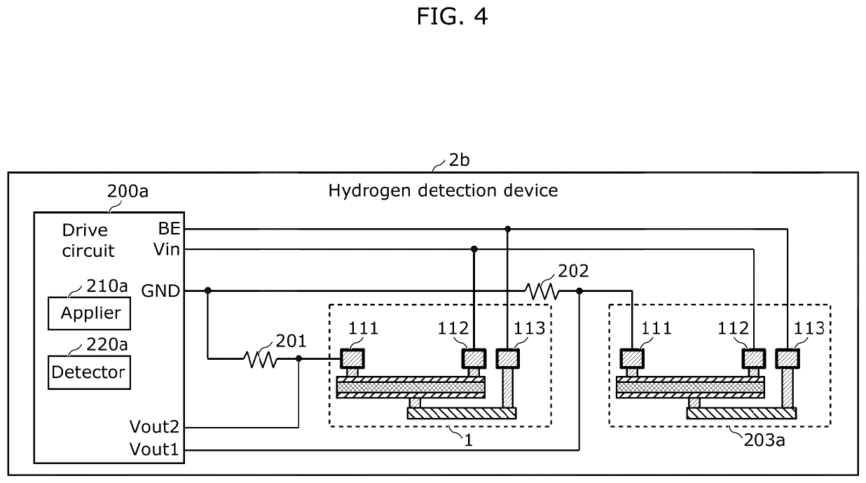

FIG. 4 is a block diagram illustrating a configuration example of a hydrogen detection device that includes a drive circuit and a hydrogen sensor and performs a hydrogen detection method using a bridge circuit according to another form of Embodiment 1. Hydrogen detection device 2b as illustrated in FIG. 4 includes, in place of resistor 203 in hydrogen detection device 2a, reference element 203a that is similar to hydrogen sensor 1 in configuration and does not include opening 106a.

Since opening 106a is not formed in reference element 203a, hydrogen does not cause a change in resistance. A change in resistance caused by a change in the ambient temperature is the same as that of hydrogen sensor 1, and thus differential voltage dV (=Vout2−Vout1) between voltage Vout2 at the connection terminal of hydrogen sensor 1 and resistor 201 and voltage Vout1 at the connection terminal of resistor 202 and reference element 203a, or the amount of change in differential voltage, can cancel the impact of the ambient temperature, enabling more accurate detection of hydrogen concentration.

[1.3 Hydrogen Detection Method according to Embodiment 1 and Experimental Data]

Next, the hydrogen detection method according to Embodiment 1 will be described using a hydrogen detection method according to a comparative example and experimental data.

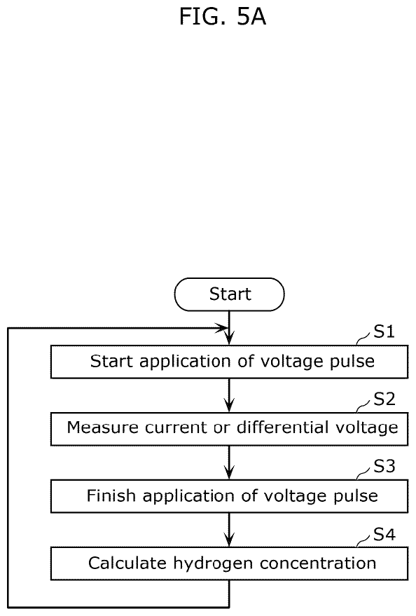

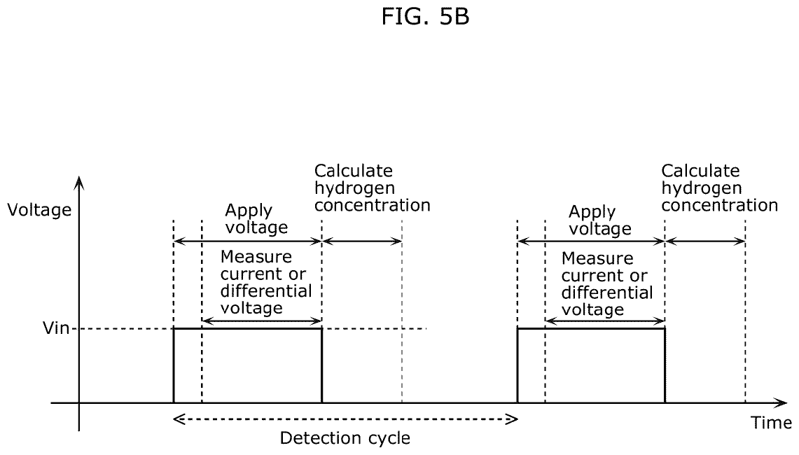

FIG. 5A is a flowchart illustrating the hydrogen detection method according to the comparative example. FIG. 5B is a diagram illustrating a voltage application pattern of the hydrogen detection method according to the comparative example illustrated in FIG. 5A when time is put on the horizontal axis. As illustrated in FIG. 5A, with the hydrogen detection method according to the comparative example, first, applier 210 of drive circuit 200 or applier 210a of drive circuit 200a starts passing current by applying a voltage pulse so that a predetermined current flows between first terminal 111 and second terminal 112 of hydrogen sensor 1 (S1). Next, while maintaining the above state, in the case of hydrogen detection device 2, detector 220 measures the current flowing between first terminal 111 and second terminal 112 or an amount of change in the current flowing between first terminal 111 and second terminal 112, whereas in the case of the bridge circuit as illustrated in hydrogen detection device 2a and hydrogen detection device 2b, detector 220a measures the differential voltage or an amount of change in the differential voltage (S2). Thereafter, the voltage application is finished (S3), and detector 220 or 220a calculates the hydrogen concentration using a predetermined conversion equation between the amount of change in differential voltage and hydrogen concentration (S4). Drive circuit 200 or 200a detects the hydrogen concentrations by repeating steps S1 through S4 at a constant cycle of, for example, 0.1 seconds to several seconds as illustrated in FIG. 5B.

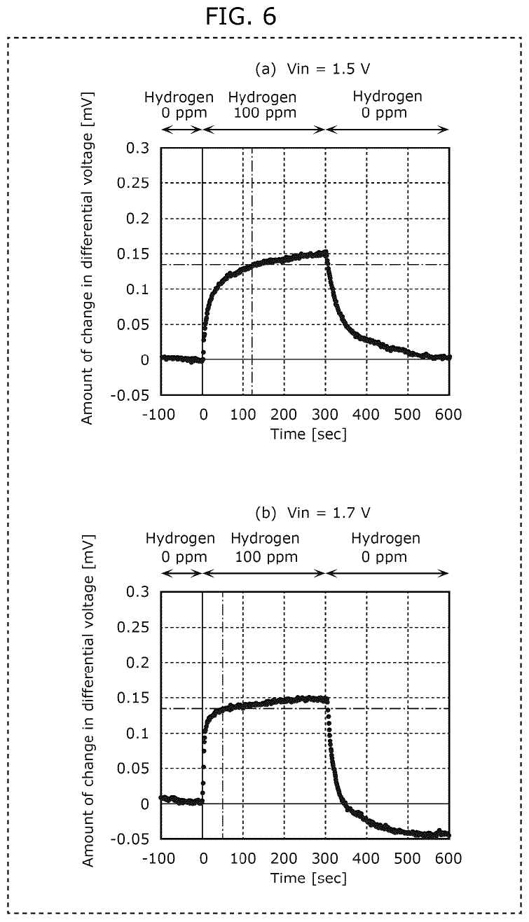

FIG. 6 is a diagram illustrating experimental results obtained by performing the hydrogen detection method according to the comparative example illustrated in FIG. 5A and FIG. 5B. This diagram illustrates the results of measurement of the amount of change in differential voltage dV (=Vout2−Vout1) measured in one-second cycles by hydrogen detection device 2b illustrated in FIG. 4 using the hydrogen detection method according to the comparative example illustrated in FIG. 5A and FIG. 5B when the hydrogen concentration is 0 ppm in the initial state (before time 0 [sec]), then hydrogen detection device 2b is exposed to a gas having a hydrogen concentration of 100 ppm (from time 0 [sec] to time 300 [sec]), and the hydrogen concentration is reset to 0 ppm (from time 300 [sec] onward).

In more detail, part (a) of FIG. 6 illustrates the amount of change in differential voltage dV (=Vout2−Vout1) when Vin=1.5 V, and part (b) of FIG. 6 illustrates the amount of change in differential voltage dV when Vin=1.7 V. Note that third terminal 113 is in a floating state and no current is passed between third terminal 113 and first terminal 111 of hydrogen sensor 1 or between third terminal 113 and second terminal 112 of hydrogen sensor 1. Resistors 201 and 202 of hydrogen detection device 2b are both set to 28Ω.

Focusing on the reaction time in part (a) of FIG. 6, the differential voltage slowly increased after the exposure to the gas having a hydrogen concentration of 100 ppm from time 0 [sec], and it took about 120 seconds to reach 90% of the maximum amount of change.

Part (b) of FIG. 6 illustrates the amount of change in differential voltage dV when applied voltage Vin is increased from 1.5 V to 1.7 V to further enhance the reaction rate. In part (b) of FIG. 6, it took about 50 seconds to reach 90% of the maximum amount of change after the exposure to the gas having a hydrogen concentration of 100 ppm from time 0 [sec], indicating that an increase in the applied voltage led to improvement in the reaction rate. This is because the amount of heat generated by current has increased, accelerating the reaction to hydrogen at exposed portion 106e of second electrode 106. However, it was found that, in part (b) of FIG. 6, when the hydrogen concentration is reset to 0 ppm after the exposure to the gas having a hydrogen concentration of 100 ppm for 300 seconds, the amount of change in differential voltage does not return to the same amount as in the initial state (before time 0 [sec]) but shifts to the negative side. That is to say, every time a gas containing hydrogen is detected, a change occurs in the base state in which the differential voltage can be converted to a hydrogen concentration of substantially 0 ppm, meaning that the hydrogen concentration cannot be accurately detected from the magnitude of a change in differential voltage.

A detailed analysis of this phenomenon revealed that flow of a large current through hydrogen sensor 1 due to application of a high voltage changes the resistance state to a high resistance state. It was also found that continuing to pass the large current leads to element destruction of hydrogen sensor 1. In other words, this change in the resistance state means element deterioration. Although the element deterioration of hydrogen sensor 1 can be inhibited to some extent by reducing the differential voltage measurement time, reduction of the measurement time, i.e., reduction of the conversion time of an AD converter included in drive circuit 200a, leads to a decrease in the accuracy of the measurement of the differential voltage. For example, it becomes impossible to maintain the accuracy of the measurement of a differential voltage corresponding to a hydrogen concentration difference of 0.1%.

One object of the present disclosure is to, for example, achieve a high reaction rate for a gas containing hydrogen at a low concentration of 100 ppm, while maintaining the base state in which the differential voltage can be converted to a hydrogen concentration of substantially 0 ppm before and after the reaction.

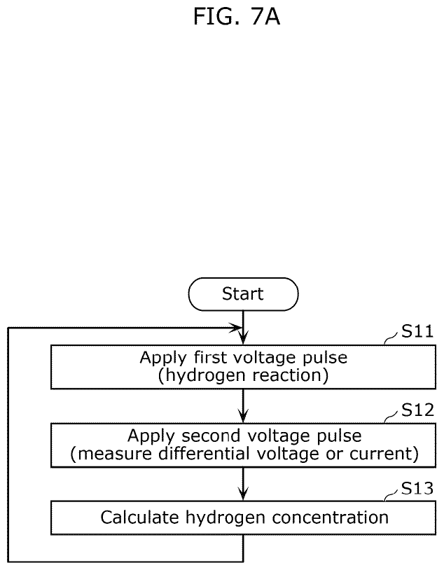

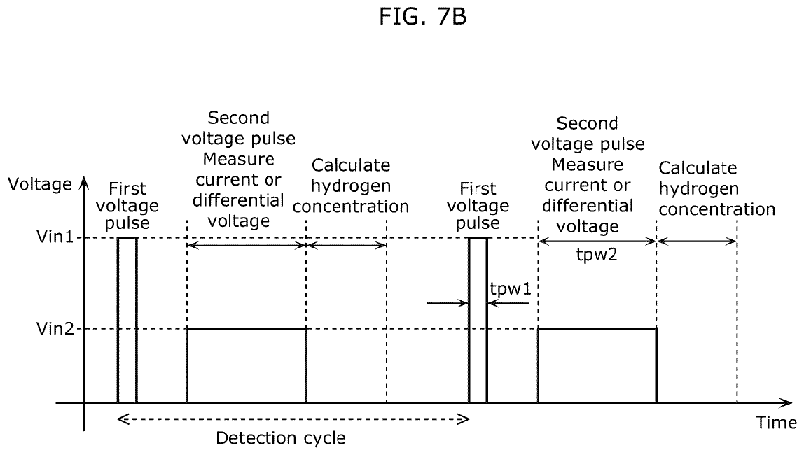

FIG. 7A is a flowchart illustrating the hydrogen detection method according to Embodiment 1. FIG. 7B is a diagram illustrating a voltage application pattern of the hydrogen detection method illustrated in FIG. 7A when time is put on the horizontal axis. As illustrated in FIG. 7A, with the hydrogen detection method according to Embodiment 1, first, applier 210 of drive circuit 200 or applier 210a of drive circuit 200a applies the first voltage pulse so that a predetermined current flows between first terminal 111 and second terminal 112 of hydrogen sensor 1 (S11). This causes a current of, for example, several milliamperes to several tens of milliamperes to flow through exposed portion 106e of second electrode 106 of hydrogen sensor 1, thereby decreasing the resistance of hydrogen sensor 1 when hydrogen sensor 1 is in contact with a gas containing hydrogen. Step S11 corresponds to applying the first voltage pulse between first terminal 111 and second terminal 112 to cause a chemical reaction of metal oxide layer 104 to hydrogen.

Note that pulse width (hereinafter also simply referred to as “width”) tpw1 of the first voltage pulse may be 1 millisecond or less so as not to cause element deterioration of hydrogen sensor 1.

Next, while the change in resistance state of hydrogen sensor 1 is maintained by the application of the first voltage pulse, applier 210 or 210a applies the second voltage pulse, and in the case of hydrogen detection device 2, detector 220 measures the current flowing between first terminal 111 and second terminal 112 or an amount of change in the current flowing between first terminal 111 and second terminal 112, whereas in the case of the bridge circuit as illustrated in hydrogen detection device 2a and hydrogen detection device 2b, detector 220a measures the differential voltage or an amount of change in the differential voltage (S12). Step S12 corresponds to applying the second voltage pulse between first terminal 111 and second terminal 112 after the applying of the first voltage pulse, to detect a change in resistance between first terminal 111 and second terminal 112.

Width tpw2 of the second voltage pulse corresponds to conversion time of the AD converter included in drive circuit 200 or 200a and may be 100 microseconds or greater to maintain the accuracy of the measurement of the current or differential voltage. Amplitude Vin2 of the second voltage pulse may be smaller than amplitude Vin1 of the first voltage pulse so that the hydrogen reaction becomes smaller than the hydrogen reaction caused by the first voltage pulse or that there is no reaction to hydrogen. Note that the time between the end of the application of the first voltage pulse and the start of the application of the second voltage pulse may be 100 milliseconds or less.

Thereafter, detector 220 or 220a calculates the hydrogen concentration using a predetermined conversion equation between the amount of change in differential voltage and hydrogen concentration (S13). Drive circuit 200 or 200a detects the hydrogen concentrations by repeating steps S11 through S13 in FIG. 7A at a constant cycle of, for example, 0.1 seconds to several seconds as illustrated in FIG. 7B.

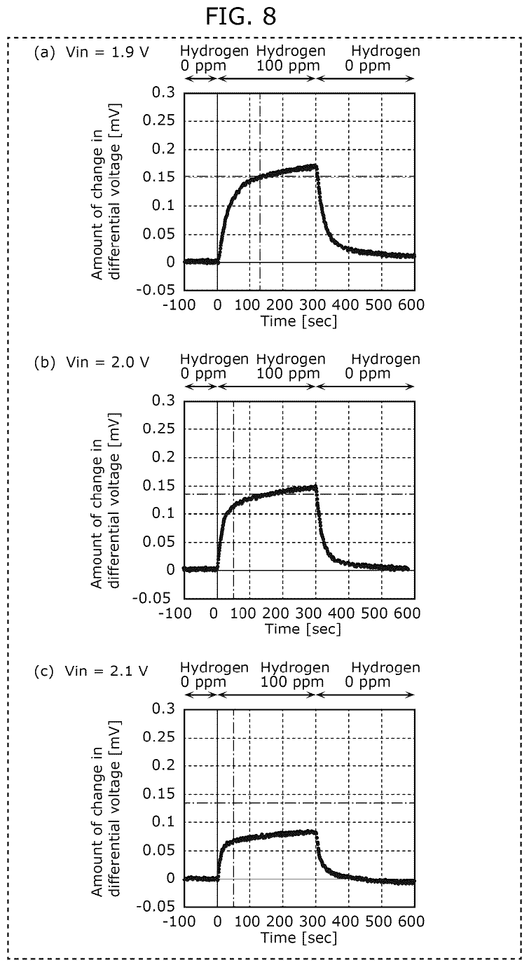

FIG. 8 is a diagram illustrating experimental results (an amount of change in differential voltage dV) on dependence on the amplitude of the first voltage pulse in the hydrogen detection method according to Embodiment 1. In more detail, FIG. 8 illustrates the results of measurement of the amount of change in differential voltage dV (=Vout2−Vout1) measured in one-second cycles by hydrogen detection device 2b illustrated in FIG. 4 using the hydrogen detection method according to Embodiment 1 when the hydrogen concentration is 0 ppm in the initial state (before time 0 [sec]), then hydrogen detection device 2b is exposed to a gas having a hydrogen concentration of 100 ppm (from time 0 [sec] to time 300 [sec]), and the hydrogen concentration is reset to 0 ppm (from time 300 [sec] onward). Note that in any of the conditions, pulse width tpw1 of the first voltage pulse is 20 microseconds, amplitude Vin2 of the second voltage pulse is 0.7 V, and pulse width tpw2 of the second voltage pulse is 2 milliseconds. Third terminal 113 is in a floating state and no current is passed between third terminal 113 and first terminal 111 of hydrogen sensor 1 or between third terminal 113 and second terminal 112 of hydrogen sensor 1. Resistors 201 and 202 of hydrogen detection device 2b are both set to 28Ω.

In more detail, part (a) of FIG. 8 illustrates the amount of change in differential voltage dV (=Vout2−Vout1) when amplitude Vin1 of the first voltage pulse is 1.9 V, part (b) of FIG. 8 illustrates the amount of change in differential voltage dV when amplitude Vin1 of the first voltage pulse is 2.0 V, and part (c) of FIG. 8 illustrates the amount of change in differential voltage dV (=Vout2−Vout1) when amplitude Vin1 of the first voltage pulse is 2.1 V.

Parts (a) through (c) of FIG. 8 show that it took about 130 seconds, about 110 seconds, and about 80 seconds, respectively, to reach 90% of the maximum amount of change after the exposure to the gas having a hydrogen concentration of 100 ppm from time 0 [sec], indicating that an increase in amplitude Vin1 of the first voltage pulse causes an increase in the reaction rate. Furthermore, they also show that even when the hydrogen concentration is reset to 0 ppm after the exposure to the gas having a hydrogen concentration of 100 ppm for 300 seconds, the amount of change in differential voltage returns to the same amount as in the initial state (before time 0 [sec]) and the phenomenon of a shift to the negative side does not occur. In other words, the base state is stable.

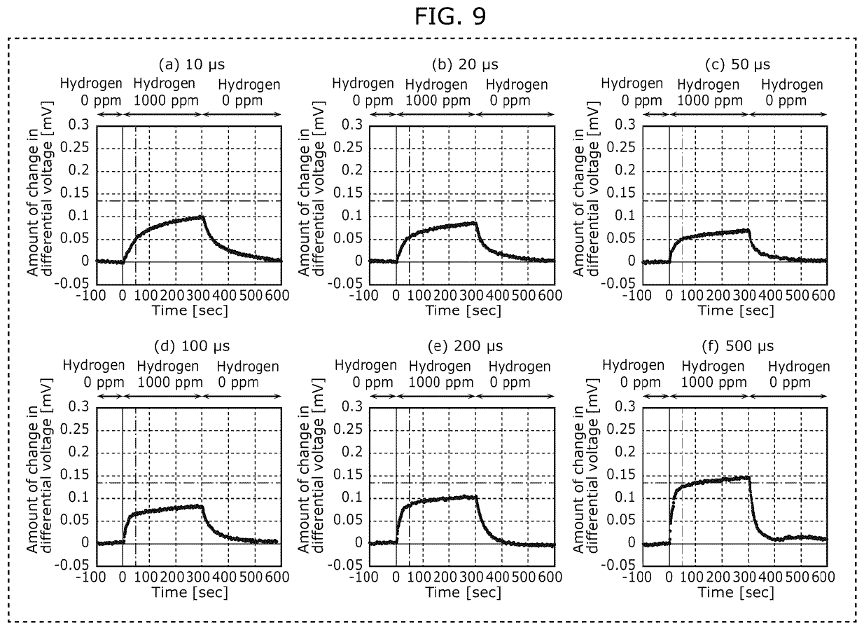

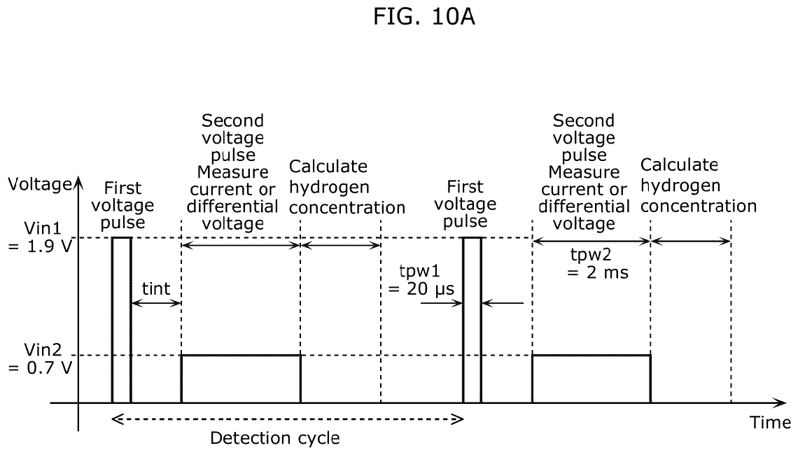

FIG. 9 is a diagram illustrating experimental results (an amount of change in differential voltage dV) on dependence on the pulse width of the first voltage pulse in the hydrogen detection method according to Embodiment 1. In more detail, FIG. 9 illustrates the results of measurement of the amount of change in differential voltage dV (=Vout2−Vout1) measured in one-second cycles by hydrogen detection device 2b illustrated in FIG. 4 using the hydrogen detection method according to Embodiment 1 when the hydrogen concentration is 0 ppm in the initial state (before time 0 [sec]), then hydrogen detection device 2b is exposed to a gas having a hydrogen concentration of 1000 ppm (from time 0 [sec] to time 300 [sec]), and the hydrogen concentration is reset to 0 ppm (from time 300 [sec] onward). This diagram illustrates the results obtained by setting width tpw1 of the first voltage pulse to various values. Note that in any of the conditions illustrated in parts (a) through (f) of FIG. 9, amplitude Vin1 of the first voltage pulse is 1.9 V, amplitude Vin2 of the second voltage pulse is 0.7 V, and the pulse width of the second voltage pulse is 2 milliseconds. Third terminal 113 is in a floating state and no current is passed between third terminal 113 and first terminal 111 of hydrogen sensor 1 or between third terminal 113 and second terminal 112 of hydrogen sensor 1. Resistors 201 and 202 of hydrogen detection device 2b are both set to 28Ω.

Part (a) of FIG. 9 illustrates the amount of change in differential voltage dV (=Vout2−Vout1) when width tpw1 of the first voltage pulse is 10 microseconds, part (b) of FIG. 9 illustrates the amount of change in differential voltage dV when width tpw1 of the first voltage pulse is 20 microseconds, part (c) of FIG. 9 illustrates the amount of change in differential voltage dV (=Vout2−Vout1) when width tpw1 of the first voltage pulse is 50 microseconds, part (d) of FIG. 9 illustrates the amount of change in differential voltage dV (=Vout2−Vout1) when width tpw1 of the first voltage pulse is 100 microseconds, part (e) of FIG. 9 illustrates the amount of change in differential voltage dV when width tpw1 of the first voltage pulse is 200 microseconds, and part (f) of FIG. 9 illustrates the amount of change in differential voltage dV (=Vout2−Vout1) when width tpw1 of the first voltage pulse is 500 microseconds.

Parts (a) through (f) of FIG. 9 show that it took about 160 seconds, about 130 seconds, about 110 seconds, about 100 seconds, about 75 seconds, and about 40 seconds, respectively, to reach 90% of the maximum amount of change after the exposure to the gas having a hydrogen concentration of 1000 ppm from time 0 [sec], indicating that an increase in the width of the first voltage pulse causes an increase in the reaction rate. Furthermore, they also show that even when the hydrogen concentration is reset to 0 ppm after the exposure to the gas having a hydrogen concentration of 1000 ppm for 300 seconds, the amount of change in differential voltage returns to the same amount as in the initial state (before time 0 [sec]) and the phenomenon of a shift to the negative side does not occur. In other words, the base state is stable.

The results in FIG. 8 and FIG. 9 show that by controlling amplitude Vin1 and pulse width tpw1 of the first voltage pulse, the rate of reaction to a gas containing hydrogen can be controlled while inhibiting deterioration of hydrogen sensor 1 and maintaining the state in which hydrogen concentration is 0 ppm. That is to say, the hydrogen concentration can be accurately calculated based on the magnitude of the amount of change in differential voltage.

Note that the first voltage pulse is applied once in Embodiment 1, but may be applied twice or more. When the first voltage pulse is applied twice or more, the amplitude of each first voltage pulse may be changed within a range greater than the second voltage pulse.

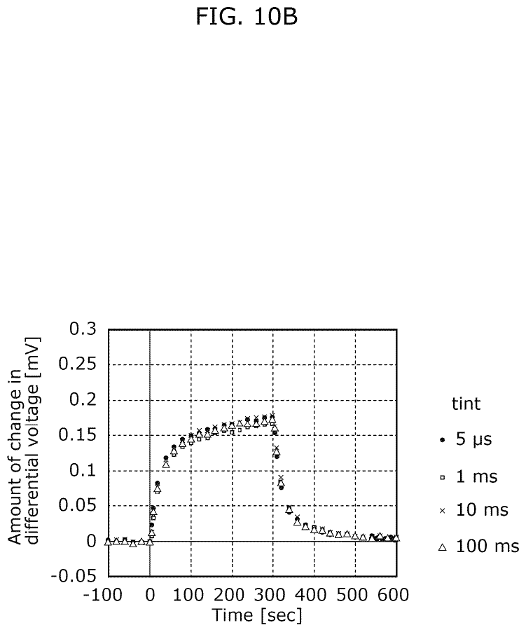

Next, the following describes an experimental result on time between the end of application of the first voltage pulse and the start of application of the second voltage pulse. FIG. 10A is a timing diagram for describing time tint between the end of application of the first voltage pulse and the start of application of the second voltage pulse. FIG. 10B is a diagram illustrating an experimental result (an amount of change in differential voltage dV) obtained when time tint in FIG. 10A is changed. In more detail, FIG. 10B illustrates the result of measurement of the amount of change in differential voltage dV (=Vout2−Vout1) measured in one-second cycles by hydrogen detection device 2b illustrated in FIG. 4 using the hydrogen detection method according to Embodiment 1 when the hydrogen concentration is 0 ppm (before time 0 [sec]) in the initial state, then hydrogen detection device 2b is exposed to a gas having a hydrogen concentration of 100 ppm (from time 0 [sec] to time 300 [sec]), and the hydrogen concentration is reset to 0 ppm (from time 300 [sec] onward), while changing time tint between the end of application of the first voltage pulse and the start of application of the second voltage pulse to 5 microseconds, 1 millisecond, 10 milliseconds, and 100 milliseconds. Note that in any of the conditions (the experiment with each time tint), amplitude Vin1 of the first voltage pulse is 1.9 V, pulse width tpw1 of the first voltage pulse is 20 microseconds, amplitude Vin2 of the second voltage pulse is 0.7 V, and pulse width tpw2 of the second voltage pulse is 2 milliseconds (see FIG. 10A). Third terminal 113 is in a floating state and no current is passed between third terminal 113 and first terminal 111 of hydrogen sensor 1 or between third terminal 113 and second terminal 112 of hydrogen sensor 1. Resistors 201 and 202 of hydrogen detection device 2b are both set to 28Ω.

The result illustrated in FIG. 10B shows that the amount of change in differential voltage dV is almost independent of time tint between the end of application of the first voltage pulse and the start of application of the second voltage pulse. That is to say, the result shows that the change in resistance state of hydrogen sensor 1 caused by the application of the first voltage pulse is maintained for at least 100 milliseconds during exposure to a gas containing hydrogen.

As described above, the hydrogen detection method according to the present embodiment is a hydrogen detection method that is performed using hydrogen sensor 1 that includes: metal oxide layer 104; second electrode 106 that is in surface contact with metal oxide layer 104; first terminal 111 connected to second electrode 106; and second terminal 112 connected to second electrode 106. The hydrogen detection method includes: applying a first voltage pulse between first terminal 111 and second terminal 112 to cause a chemical reaction of metal oxide layer 104 to hydrogen; and applying a second voltage pulse between first terminal 111 and second terminal 112 after the applying of the first voltage pulse, to detect a change in resistance between first terminal 111 and second terminal 112. The amplitude of the second voltage pulse is smaller than the amplitude of the first voltage pulse.

Accordingly, the resistance between first terminal 111 and second terminal 112 returns to the same level as in the initial state (base state) even when hydrogen is no longer present after the exposure to hydrogen, thus stabilizing the base state and realizing more accurate hydrogen concentration detection than conventional techniques.

Here, the pulse width of the first voltage pulse may be 1 millisecond or less. Accordingly, by increasing the width of the first voltage pulse within this pulse width range, the reaction rate of the hydrogen sensor can be increased and the stability of the base state of the hydrogen sensor can be ensured.

Also, the pulse width of the second voltage pulse may be 100 microseconds or greater. Accordingly, the conversion time of the AD converter included in drive circuit 200 or 200a is secured, and high measurement accuracy of the detection of a change in resistance between first terminal 111 and second terminal 112 is maintained.

Also, the first voltage pulse may be applied twice or more. Accordingly, the detection can be stabilized by, for example, averaging the detection results.

Also, time between an end of application of the first voltage pulse and a start of application of the second voltage pulse may be 100 milliseconds or less. This reduces dependence on the time between the end of application of the first voltage pulse and the start of application of the second voltage pulse, thus enabling stable hydrogen detection.

Also, hydrogen sensor 1 may be one of four resistors included in a bridge circuit, and in the applying of the second voltage pulse, the change in resistance between first terminal 111 and second terminal 112 may be detected by measuring a differential voltage between predetermined two points in the bridge circuit or an amount of change in the differential voltage. Accordingly, it is possible to realize a highly sensitive hydrogen detection method performed using a bridge circuit.

The drive circuit according to the present embodiment is drive circuit 200 or 200a that drives hydrogen sensor 1 that includes: metal oxide layer 104; second electrode 106 that is in surface contact with metal oxide layer 104; first terminal 111 connected to second electrode 106; and second terminal 112 connected to second electrode 106. The drive circuit includes: applier 210 or 210a that applies a first voltage pulse between first terminal 111 and second terminal 112 to cause a chemical reaction of metal oxide layer 104 to hydrogen, and then applies a second voltage pulse between first terminal 111 and second terminal 112; and detector 220 or 220a that detects resistance between first terminal 111 and second terminal 112 while the second voltage pulse is applied between first terminal 111 and second terminal 112. The amplitude of the second voltage pulse is smaller than the amplitude of the first voltage pulse.

The hydrogen detection device according to the present embodiment includes: hydrogen sensor 1 that includes metal oxide layer 104, second electrode 106 that is in surface contact with metal oxide layer 104, first terminal 111 connected to second electrode 106, and second terminal 112 connected to second electrode 106; and above-described drive circuit 200 or 200a that drives hydrogen sensor 1.

With such a drive circuit and hydrogen detection device, the resistance between first terminal 111 and second terminal 112 returns to the same level as in the initial state (base state) even when hydrogen is no longer present after the exposure to hydrogen, thus stabilizing the base state and realizing more accurate hydrogen concentration detection than conventional techniques.

### Embodiment 2

Next, a hydrogen detection method according to Embodiment 2 will be described.

### [2.1 Dependence on Hydrogen Concentration]

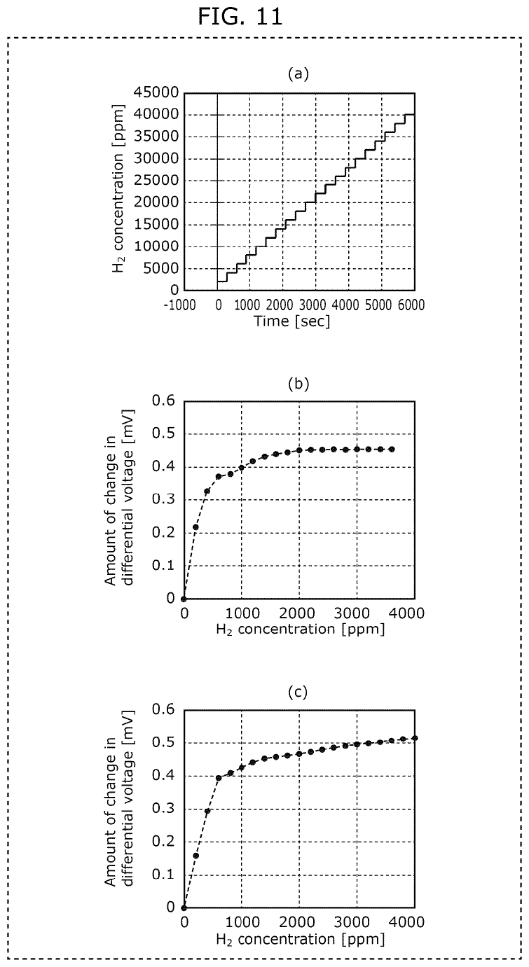

First, the following describes experimental results that have led to conception of the hydrogen detection method according to Embodiment 2. FIG. 11 is a diagram for describing experiments related to the hydrogen detection method according to Embodiment 2. In more detail, part (a) of FIG. 11 illustrates change over time in hydrogen concentration in an experiment conducted using the hydrogen detection method according to Embodiment 2, part (b) of FIG. 11 illustrates an experimental result (an amount of change in differential voltage dV) obtained using a hydrogen detection method according to a comparative example, and part (c) of FIG. 11 illustrates an experimental result (an amount of change in differential voltage dV) obtained using the hydrogen detection method according to Embodiment 2. In the experiments, hydrogen detection device 2b as in Embodiment 1 measured the amount of change in differential voltage when the hydrogen concentration was increased in 2000-ppm increments every 300 seconds from time 0 [sec] as illustrated in part (a) of FIG. 11.

The average value of the amount of change in differential voltage in a time period between time 200 [sec] and time 210 [sec] is plotted for each hydrogen concentration (in parts (b) and (c) of FIG. 11).

Part (b) of FIG. 11 illustrates the amount of change in differential voltage corresponding to each hydrogen concentration obtained in the experiment when amplitude Vin1 of the first voltage pulse is 1.9 V, pulse width tpw1 of the first voltage pulse is 100 microseconds, amplitude Vin2 of the second voltage pulse is 0.7 V, and pulse width tpw2 of the second voltage pulse is 2 milliseconds.

The result in part (b) of FIG. 11 shows that the amount of change in differential voltage is saturated when the hydrogen concentration is 20000 ppm (2%) or greater. This means that the hydrogen concentration cannot be accurately detected at a hydrogen concentration of 2% or greater. Embodiment 2 describes a method of detecting not only low-concentrated hydrogen but also a wide range of hydrogen concentrations from 0% to 4% inclusive, in contrast to the hydrogen detection method according to Embodiment 1. Part (c) of FIG. 11 will be described later.

### [2.2 Hydrogen Detection Method of Embodiment 2]

With the hydrogen detection method according to Embodiment 2, drive circuit 200 or 200a changes at least one of the amplitude of the first voltage pulse, the width of the first voltage pulse, or the amplitude of the second voltage pulse, according to at least two hydrogen concentration ranges (i.e., the ranges of hydrogen concentration to be detected).

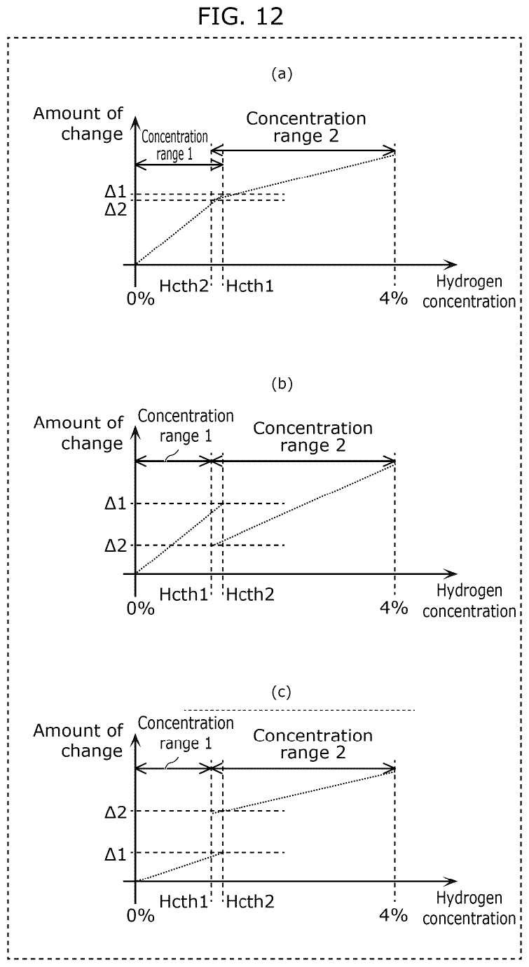

FIG. 12 is a diagram illustrating relationships between hydrogen concentration and an amount of change in differential voltage in the hydrogen detection method according to Embodiment 2. Illustrated here are examples in which setting conditions for the first voltage pulse and the second voltage pulse are changed according to each of two concentration ranges obtained by dividing a hydrogen concentration range of from 0% to 4% inclusive into concentration range 1 of from 0% to Hcth1 inclusive and concentration range 2 of from Htch2 to 4% inclusive, for example (parts (a) through (c) of FIG. 12). Here, Hcth1≥Hcth2, and the concentration ranges may be set to partially overlap as illustrated in part (a) of FIG. 12. When Δ1 denotes an amount of change in current or an amount of change in differential voltage in the case of hydrogen concentration Htch1 in the pulse setting condition for concentration range 1, and Δ2 denotes an amount of change in current or an amount of change in differential voltage in the case of hydrogen concentration Htch2 in the pulse setting condition for concentration range 2, the relationship between Δ1 and Δ2 is not limited (see parts (a) and (b) of FIG. 12 and part (c) of FIG. 12). The amount of change in current or the amount of change in differential voltage in concentration range 1 and the amount of change in current or the amount of change in differential voltage in concentration range 2 may continuously vary with respect to the hydrogen concentration as illustrated in part (a) of FIG. 12 or may discontinuously vary as illustrated in parts (b) and (c) of FIG. 12.

If (hydrogen concentration in concentration range 1)<(hydrogen concentration in concentration range 2) now, in concentration range 1, amplitude Vin1 of the first voltage pulse is set to Vin1L, pulse width tpw1 of the first voltage pulse is set to tpw1L, and amplitude Vin2 of the second voltage pulse is set to Vin2L, whereas in concentration range 2, amplitude Vin1 of the first voltage pulse is set to Vin1H, pulse width tpw1 of the first voltage pulse is set to tpw1H, and amplitude Vin2 of the second voltage pulse is set to Vin2H.

Here, Vin1L>Vin2L and Vin1H>Vin2H are satisfied, and at least one of Vin1L>Vin1H, tpw1L>tpw1H, or Vin2L>Vin2H may be further satisfied. Width tpw2 of the second voltage pulse corresponds to conversion time of the AD converter included in drive circuit 200 or 200a and is set in advance, and may be 1 millisecond or greater to maintain the accuracy of the measurement of the current or differential voltage.

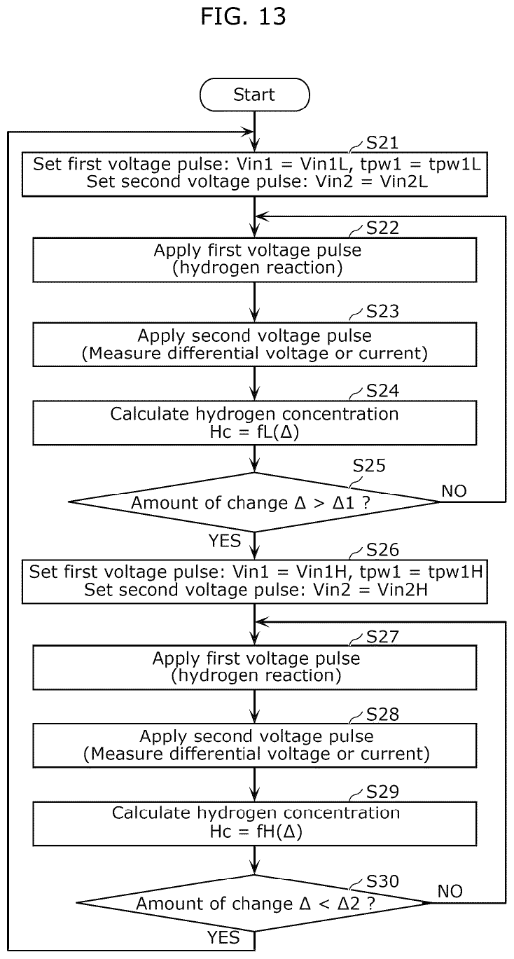

FIG. 13 is a flowchart illustrating the hydrogen detection method according to Embodiment 2.

In the hydrogen detection method according to Embodiment 2, first, before the detection starts, the pulse setting condition for concentration range 1, i.e., the lowest hydrogen concentration range, is set. That is to say, amplitude Vin1 of the first voltage pulse is set to Vin1L, pulse width tpw1 of the first voltage pulse is set to tpw1L, and amplitude Vin2 of the second voltage pulse is set to Vin2L (S21). Thereafter, applier 210 or 210a applies the first voltage pulse so that a predetermined current flows between first terminal 111 and second terminal 112 of hydrogen sensor 1 (S22). With this, a current of, for example, several milliamperes to several tens of milliamperes flows through exposed portion 106e of second electrode 106 of hydrogen sensor 1, thereby decreasing the resistance of hydrogen sensor 1 when hydrogen sensor 1 is in contact with a gas containing hydrogen.

Note that width tpw1 of the first voltage pulse may be 1 millisecond or less so as not to cause element deterioration of hydrogen sensor 1.

Next, while the change in resistance state of hydrogen sensor 1 is maintained by the application of the first voltage pulse, applier 210 or 210a applies the second voltage pulse, and in the case of hydrogen detection device 2, detector 220 measures the current flowing between first terminal 111 and second terminal 112 or an amount of change in the current flowing between first terminal 111 and second terminal 112, whereas in the case of the bridge circuit as illustrated in hydrogen detection device 2a and hydrogen detection device 2b, detector 220a measures the differential voltage or an amount of change in the differential voltage (S23). Note that amplitude Vin2 of the second voltage pulse may be smaller than amplitude Vin1 of the first voltage pulse so that the hydrogen reaction becomes smaller than the hydrogen reaction caused by the first voltage pulse or that there is no reaction to hydrogen. The time between the end of the application of the first voltage pulse and the start of the application of the second voltage pulse may be 100 milliseconds or less.

Thereafter, detector 220 or 220a calculates the hydrogen concentration using a predetermined conversion equation between the amount of change in concentration range 1 and hydrogen concentration (S24).

Next, detector 220 or 220a determines whether the measured amount of change in current or amount of change in differential voltage is greater than predetermined threshold Δ1 (S25). Here, when the amount of change in current or the amount of change in differential voltage is Δ1 or less (NO in S25), the pulse condition setting is not changed and the next measurement cycle starts from step S22, whereas when the amount of change in current or the amount of change in differential voltage is greater than Δ1 (YES in S25), the next measurement cycle starts from step S26 and applier 210 or 210a changes the pulse setting condition to the pulse setting condition for concentration range 2. Thereafter, applier 210 or 210a applies the first voltage pulse so that a predetermined current flows between first terminal 111 and second terminal 112 of hydrogen sensor 1 (S27). With this, a current of, for example, several milliamperes to several tens of milliamperes flows through exposed portion 106e of second electrode 106 of hydrogen sensor 1, thereby decreasing the resistance of hydrogen sensor 1 when hydrogen sensor 1 is in contact with a gas containing hydrogen.

Note that width tpw1 of the first voltage pulse may be 1 millisecond or less so as not to cause element deterioration of hydrogen sensor 1.

Next, while the change in resistance state of hydrogen sensor 1 is maintained by the application of the first voltage pulse, applier 210 or 210a applies the second voltage pulse, and in the case of hydrogen detection device 2, detector 220 measures the current flowing between first terminal 111 and second terminal 112 or an amount of change in the current flowing between first terminal 111 and second terminal 112, whereas in the case of the bridge circuit as illustrated in hydrogen detection device 2a and hydrogen detection device 2b, detector 220a measures the differential voltage or an amount of change in the differential voltage (S28). Note that amplitude Vin2 of the second voltage pulse may be smaller than amplitude Vin1 of the first voltage pulse so that the hydrogen reaction becomes smaller than the hydrogen reaction caused by the first voltage pulse or that there is no reaction to hydrogen. The time between the end of the application of the first voltage pulse and the start of the application of the second voltage pulse may be 100 milliseconds or less.

Thereafter, detector 220 or 220a calculates the hydrogen concentration using a predetermined conversion equation between the amount of change in concentration range 2 and the hydrogen concentration (S29).

Next, detector 220 or 220a determines whether the measured amount of change in current or amount of change in differential voltage is less than predetermined threshold Δ2 (S30). Here, when the amount of change in current or the amount of change in differential voltage is Δ2 or greater (NO in S30), the pulse condition setting is not changed and the next measurement cycle starts from step S27, whereas when the amount of change in current or the amount of change in differential voltage is less than Δ2 (YES in S30), the next measurement cycle starts from step S21 and applier 210 or 210a changes the pulse setting condition to the pulse setting condition for concentration range 1. From then onward, the hydrogen concentration detection is repeatedly performed in cycles of, for example, 0.1 seconds to several seconds while changing the pulse setting condition according to the concentration range in the same manner.

Note that steps S21 and S26 correspond to the changing of at least one of the amplitude of the first voltage pulse, the pulse width of the first voltage pulse, or the amplitude of the second voltage pulse, according to a range of hydrogen concentration to be detected.

### [2.3 Experimental Data]

Next, operation of hydrogen sensor 1 according to Embodiment 2 will be described using experimental data.

Part (c) of FIG. 11 is a diagram illustrating experimental data obtained by performing the hydrogen detection method according to Embodiment 2. Illustrated here is the result of measurement, performed by hydrogen detection device 2b as in Embodiment 1, of the amount of change in differential voltage when the hydrogen concentration was increased in 2000-ppm increments every 300 seconds from time 0 [sec] as illustrated in part (a) of FIG. 11.

The average value of the amount of change in differential voltage in a time period between time 200 [sec] and time 210 [sec] is plotted for each hydrogen concentration.

The setting conditions for the first voltage pulse and the second voltage pulse are set as follows. Concentration range 1 is set to 0 to 4000 ppm inclusive and concentration range 2 is set to 4000 ppm to 4% inclusive. Here, the value of Δ1 and the value of Δ2 are both 0.3 mV. In concentration range 1, amplitude Vin1L of the first voltage pulse is set to 1.9 V, pulse width tpw1L of the first voltage pulse is set to 100 microseconds, and amplitude Vin2L of the second voltage pulse is set to 0.7 V, whereas in concentration range 2, amplitude Vin1H of the first voltage pulse is set to 1.5 V, pulse width tpw1H of the first voltage pulse is set to 100 microseconds, and amplitude Vin2H of the second voltage pulse is set to 0.7 V.

As described above, the hydrogen detection method according to the present embodiment includes the hydrogen detection method according to Embodiment 1 and further includes changing at least one of the amplitude of the first voltage pulse, the pulse width of the first voltage pulse, or the amplitude of the second voltage pulse, according to the range of hydrogen concentration to be detected. Accordingly, even when the hydrogen concentration is high, saturation of the dependence of the hydrogen sensor on hydrogen concentration is inhibited and the hydrogen concentration is detected in a wide range.

Although the hydrogen sensor, hydrogen detection method, drive circuit, and hydrogen detection device according to one or more aspects have been described based on Embodiments 1 and 2, the present disclosure is not limited to Embodiments 1 and 2. Various modifications to Embodiments 1 and 2 that are conceivable to those skilled in the art, as well as forms resulting from combinations of constituent elements of different embodiments may be included within the scope of one or more aspects, so long as such modifications and forms do not depart from the essence of the present disclosure.

For example, although the range of hydrogen concentration to be measured is divided into two in Embodiment 2, it may be divided into 3 or more.

In addition, although the lower hydrogen concentration range is measured prior to the higher hydrogen concentration range in Embodiment 2, the higher hydrogen concentration range may be measured prior to the lower hydrogen concentration range.

The hydrogen detection methods according to the above embodiments may be realized as programs executed by a processor. Such programs may be distributed by being stored in a non-transitory computer-readable recording medium such as a digital versatile disc (DVD) or may be distributed by being transferred via a communication line such as the Internet.

Although only some exemplary embodiments of the present disclosure have been described in detail above, those skilled in the art will readily appreciate that many modifications are possible in the exemplary embodiments without materially departing from the novel teachings and advantages of the present disclosure. Accordingly, all such modifications are intended to be included within the scope of the present disclosure.

### INDUSTRIAL APPLICABILITY

The hydrogen detection method, drive circuit, and hydrogen detection device according to the present disclosure can be widely used for a hydrogen detection device that stabilizes the base state and detects a wide range of hydrogen concentration, e.g., a hydrogen detection device that detects leakage of a gas containing hydrogen.

1. A hydrogen detection method that is performed using a hydrogen sensor,
the hydrogen sensor including:
a metal oxide layer;
an electrode that is in surface contact with the metal oxide layer;
a first terminal connected to the electrode; and
a second terminal connected to the electrode,
the hydrogen detection method comprising:
applying a first voltage pulse between the first terminal and the second terminal to cause a chemical reaction of the metal oxide layer to hydrogen; and
applying a second voltage pulse between the first terminal and the second terminal after the applying of the first voltage pulse, to detect a change in resistance between the first terminal and the second terminal,
wherein an amplitude of the second voltage pulse is smaller than an amplitude of the first voltage pulse.

2. The hydrogen detection method according to claim 1,
wherein a pulse width of the first voltage pulse is 1 millisecond or less.

3. The hydrogen detection method according to claim 1,
wherein a pulse width of the second voltage pulse is 100 microseconds or greater.

4. The hydrogen detection method according to claim 1,
wherein the first voltage pulse is applied twice or more.

5. The hydrogen detection method according to claim 1,
wherein time between an end of application of the first voltage pulse and a start of application of the second voltage pulse is 100 milliseconds or less.

6. The hydrogen detection method according to claim 1, further comprising:
changing at least one of the amplitude of the first voltage pulse, a pulse width of the first voltage pulse, or the amplitude of the second voltage pulse, according to a range of hydrogen concentration to be detected.

7. The hydrogen detection method according to claim 1,
wherein the hydrogen sensor is one of four resistors included in a bridge circuit, and
in the applying of the second voltage pulse, the change in resistance between the first terminal and the second terminal is detected by measuring a differential voltage between predetermined two points in the bridge circuit or an amount of change in the differential voltage.

8. A drive circuit that drives a hydrogen sensor,
the hydrogen sensor including:
a metal oxide layer;
an electrode that is in surface contact with the metal oxide layer;
a first terminal connected to the electrode; and
a second terminal connected to the electrode,
the drive circuit comprising:
an applier that applies a first voltage pulse between the first terminal and the second terminal to cause a chemical reaction of the metal oxide layer to hydrogen, and then applies a second voltage pulse between the first terminal and the second terminal; and
a detector that detects resistance between the first terminal and the second terminal while the second voltage pulse is applied between the first terminal and the second terminal,
wherein an amplitude of the second voltage pulse is smaller than an amplitude of the first voltage pulse.

9. A hydrogen detection device comprising:
a hydrogen sensor that includes a metal oxide layer, an electrode that is in surface contact with the metal oxide layer, a first terminal connected to the electrode, and a second terminal connected to the electrode; and
the drive circuit according to claim 8 that drives the hydrogen sensor.
